(19) United States
(12) Patent Application Publication
(10) Pub. No.: US 2013/0324885 A1
(43) Pub. Date: Dec. 5, 2013

(54) SYSTEM TO IMPROVE THE QUALITY OF THE BLOOD SAMPLE OBTAINED FROM INFANTS FEET AND DISPOSABLE HEATABLE BOOTY THEREOF

(71) Applicant: Jacqueline Ivonne Santos Rodriguez, Maldonado (UR)

(72) Inventor: Jacqueline Ivonne Santos Rodriguez, Maldonado (UR)

(21) Appl. No.: 13/904,137

(22) Filed: May 29, 2013

Related U.S. Application Data
(60) Provisional application No. 61/652,480, filed on May 29, 2012.

(51) Int. Cl. A61B 5/15

(52) U.S. Cl. CPC A61B 5/150076
USPC 600/573; 607/111

(57) ABSTRACT

System to improve the quality of blood sample obtained from the foot of an infant specially a newborn baby. The system includes a disposable heatable booty that allows the vessel dilation without massage, friction or pressure, and without injuries or burns. Samples of blood improve in quality and quantity, avoiding the multi-puncture of the foot of the infant, to prevent suffering on the infant and facilitating the work of the personnel performing the extraction.

### CROSS REFERENCE TO RELATED APPLICATION

This application claims priority upon U.S. provisional Patent Application No. 61/652,480 filed May 29, 2012, the whole of which is incorporated herein by reference.

### FIELD OF THE INVENTION

The invention relates to systems used to improve the quality of the capillary blood samples obtained from infants, particularly newborns.

### BACKGROUND OF THE INVENTION

In many third world countries, the so-called mandatory neonatal research has been implanted; in other countries of the world this neonatal research is voluntary. It is a method of early diagnosis of diseases, which applies especially in newborn children of forty hours old.

The neonatal screening is a method of early diagnosis of many diseases, such as phenylketonuria or hyperphenylalaninemia, adrenal hyperplasia, hypothyroidism, which can cause in the newborn mental retardation, among others, but if detected and treated early and at the appropriate time would not lead to major problems. As research progresses, it is discovered that other diseases may be detected early using this diagnosis method.

This method of diagnosis involves, in many countries, the heel puncture of the newborn, from where samples of blood are extracted, by using a Lancet.

For the correct sampling during these procedures, it is necessary for the blood to go out of the body fluidly; it is well known that the use of the Lancet does not provide the security that the blood-goes out of the body fluidly. In some regulations and instructions that are provided to the person taking the samples, they are encouraged to rub or massage the newborn's heel prior to the puncture. It is also possible to see that some of the people who take the samples they press the heel of the newborn in search of better blood samples. The most frequent is to see the person taking the samples to repeat the punctures several times in order to obtain a proper sample.

The pressure on the delicate newborn's heel may cause rupture of capillaries, hemorrhage, rupture of cartilage, contamination of the blood sample with interstitial and intracellular fluids. Rubbing or massaging the heel may cause hemolysis and other injuries that may compromise the quality of subsequent samples, in case there is a need to repeat them. Multi-puncture may be a cruel procedure resisted by the newborn baby and his/her relatives, making more difficult work for the person taking the sample.

It is well known that warming up the skin in the area to be punctured increases the capillary action by vessel dilation; this increases blood flow in the area to be punctured. Academic work, at the international level, discusses the need to warm the infant's heel in order to facilitate obtaining the blood sample.

Many devices are known in the form of bandages or disposable cloths that allow to warm the children's heels prior to the puncture. These bandages or disposable cloths can be heated, others are made of thermal materials; these cloths or bandages are applied to the area that needs to be heated and others are wrapped around the feet or ankles, or have other adjustment mechanisms. In the case of babies, if these bandages or cloths are heated in excess they can cause burns on the delicate skin of the babies, and with regards to its shape, the baby can get rid of them, because they are uncomfortable or annoying. This further complicates the work of the person taking the sample and makes necessary to perform_multi-puncture, since its use does not guarantee the proper capillary dilation in the infant foot, so the quality of the samples it is not the sought one.

Also, there are known heaters, including fragments or sectors, that are joined to each other by tape or other adjustment elements that are placed around the foot or the heel; these devices need brooches that engage into holes to avoid the patient removing them or the separation or movement of the fragments and do not fulfill its function properly. These devices are not suitable for babies, since they constantly move, which complicates its placement and on the other hand, they may be uncomfortable so the babies may try to remove them. The result is the same as in the case of bandages or cloth, that is the multi-puncture to achieve the quality looked for in the required blood samples. On the other hand, the fact that its making is complicated increases the production cost which can tempt to reuse them instead of discarding them because they are usually contaminated with fluids of patients.

To solve these problems, in the U.S. provisional application No. 61/652,480, the inventor proposed a disposable heatable booty for heating the babies' heels prior to capillary puncture.

### Object of the Invention

It is an object of the present invention to prevent the multi-puncture of the newly born, and, in general, to decrease the number of repetitions of the puncture of the newborn's heel, through providing a system to improve the quality and quantity of the blood sample, achieving efficiency.

It is another object of the invention to help achieve an early diagnosis of diseases and treatment at the proper time, if necessary.

A third object of the invention is to implement a user-friendly device for the newborn baby and their families, disposable and heatable, which facilitates the taking of the blood sample for the neonatal research and other studies that require a skin puncture. It is well known that the booty shape is more comfortable and friendly for babies since booties are one of the elements that are worn by them. On the other hand, family members do not provide resistance to place booties to bring warmth to the delicate feet of the baby.

It is a fourth object of the invention to achieve an adequate vasodilation and a greater blood supply by warming the foot of the child, especially newborns.

A fifth object of this invention is to reduce the suffering of the child during the execution of the procedure of obtaining the blood sample and improve the quality of the health service. The use of bags of hot water, microwave heatable bags, heatable bandages, rubbing, or massaging, can cause injury and burns on the delicate skin of the newborn and in other cases lesions on their cartilage, bleeding, among other possible problems.

It is a sixth object of this invention to simplify the work of the nursing staff responsible for obtaining the blood samples.

It is a seventh object of this invention to reduce the costs of necessary materials.

### Description of the Disposable Heatable Booties and Method of Manufacturing

A sterile disposable heatable booty to bring warmth to the foot of the newborn for the realization of the capillary puncture.

The booty is made of fabrics that are disposable and heatable, for example, paper fabric and a soft fabric such as, for example, the soft micro fleece or polar fleece fabric.

The body of the booty can be cut in one piece and is attached with a single seam or union, or in, for example, two pieces stitched or united to each other, for example a superior piece as a shoe upper and a lower piece that covers the bottom of the foot, the sides, and the back of the foot with a seam or union in the area of the back of the foot. The booty covers the bottom of the foot, sides, the instep of the foot, and the toes. The body of the booty is sewn or attached by other means to a cuff made of elastic fabric that gently adjusts to the ankle area of the baby. So the foot is completely covered. The cuff gently adjusts the booty to the infant ankle to avoid the infant from removing it with its natural movements.

During the testing of the prototype, it was demonstrated to provide efficiency to place inside the booty a sole made of paper fabric at the bottom of it, wrapped in polar fleece fabric, over the fabric paper sole within the wrapping of fleece fabric there are placed heatable seeds, for example, wheat. This sole with seeds wrapped in a soft cloth may be loose or tight to the bottom of the booty.

In another example of manufacturing, the fabric paper sole is replaced with a gel capsule with seeds like, for example, wheat. The capsule is wrapped in a soft cloth. The capsule wrapped in soft fabric may be loose or attached to the bottom inside of the booty.

The baby's skin does not come in contact with the seeds or with the gel capsule having the seeds, but it comes in contact with the soft fabric.

The booty measures that have proven to be appropriate during the testing of the prototype are the following: 7 cm long, 4 cm wide, 5 cm high, and weighs approximately 15 grams.

The measurement of the sole with seeds wrapped in soft fabric and the gel capsule with seeds are proportional to the above indicated measurements.

The booties can be heated in a microwave or other devices to warm them and that allow a control of the temperature to be applied. The booties are not connected to the electricity grid. The booties do not carry a device or any chemical substance that allows self-heating as a way to provide safety to the newborns.

After heated, the booties are placed at the feet of the baby or child transmitting heat to the heels producing peripheral dilation of the blood vessels. With the heat, the skin becomes red, since capillaries of the skin are dilated to increase the heat exchange area with the surface area, and as a consequence increases the blood supply.

The use of heatable seeds is recommended by health professionals, since they provide analgesia and anti-inflammatory properties that promote vasodilation. In known bags of seeds which are used for heating, for example, babies cribs, between the seeds is air. Once the pods are removed from the heating device, the presence of air between the seeds interrupts the heat transmission between them, causing a heat loss and minimizing the total volume of the condensate element of heat that are the seeds. The introduction of seeds in the gel capsule replaces the air with gel. Thus, the interruption of transmission of heat is removed and additionally increases the condensation volume of heat through the replacement of the air by the gel.

During the testing of the prototypes at the newborns' feet, highly satisfactory results were found: effective heating of the newborn's heel; injuries do not occur in the skin unlike what happens with the friction of the same; there are no burns; the blood samples significantly improve in quantity and quality; the babies and their families do not resist and, therefore, allow the trained personnel to work comfortably and efficiently.

### DESCRIPTION OF DRAWINGS

FIG. 1 shows an example of the manufacturing of a disposable heatable booty for capillary puncture;

FIG. 2 shows a cross-sectional view of lines A-A″ and B-B″;

FIG. 3 shows the disposable heatable booty illustrating the upper part which corresponds to the elastic cuff that adjusts to the ankle C, the rear part D, the front part E and the bottom part F;

FIG. 4 shows a cross-sectional view B-B″ illustrating the booty (1) made of paper fabric, the cuff (2) of the booty made of elastic fabric, sewing of the cuff (3) that it connects to the body of the booty, gel capsule (7) with seeds (9), soft fabric (8) that envelops the capsule of gel, part of the booty that goes on the instep of the foot (6);

FIG. 5 shows a cross-sectional view A-A″ showing the booty (1) made of paper fabric, cuff (2) in booty made of elastic fabric, cuff's sewing (3), uppers or upper part (5) of the booty, seam (4) that joins the shoe upper to the other part of the booty, the gel capsule (7) with seeds (9), and soft fabric (8) that envelops the gel capsule.

FIG. 6 shows a cross-sectional view B-B″ illustrating the booty (1), the cuff (2) made of elastic fabric, the sewing (3) of the cuff, the upper or upper part (5) of the booty, the gel capsule (7) with seeds (9), the soft fabric (8) that envelops the gel capsule;

FIG. 7 shows a cross-sectional view of the bottom section of the booty (1) illustrating the interior of the gel capsule (7) with seeds (9);

FIG. 8 shows a cross-sectional view B-B″ illustrating the booty (1), cuff (2) made of elastic fabric, sewing (3) of the cuff, the portion of the booty (6) that goes on the instep of the foot, seeds (9), sole (11) made of paper fabric, soft fabric (10) that surrounds the sole and seeds;

FIG. 9 shows a cross-sectional view A-A″ illustrating the booty (1), cuff (2) made of elastic fabric , sewing (3) of the cuff, uppers or upper part (5) of the booty, seam (4) that joins the shoe upper to the other part of the booty, seeds (9), sole (11) made of paper fabric, soft fabric (10) that surrounds template and seeds;

FIG. 10 shows a cross-sectional view B-B″ showing the booty (1), the cuff (2) made of fabric elastic, upper or upper part (5) of the booty, seeds (9), sole (11) made of paper fabric, soft fabric (10) that surrounds the sole and the seeds;

FIG. 11 shows a gel capsule (7) with seeds (9), soft fabric (8) that envelops the gel capsule;

FIG. 12 shows seeds (9), the sole (11) made of paper fabric, soft fabric (10) that surrounds template and seeds;

FIG. 13 shows the area (12) of the bottom of the foot of the newborn pointing the puncture zone;

FIG. 14 shows a filter paper (13) where blood samples are being deposited and drops of blood (14) obtained when the booty is not used.

FIG. 15 shows a filter paper (13) where blood samples are being deposited and drops of blood (15, 16) of larger size that are obtained when using the booty.

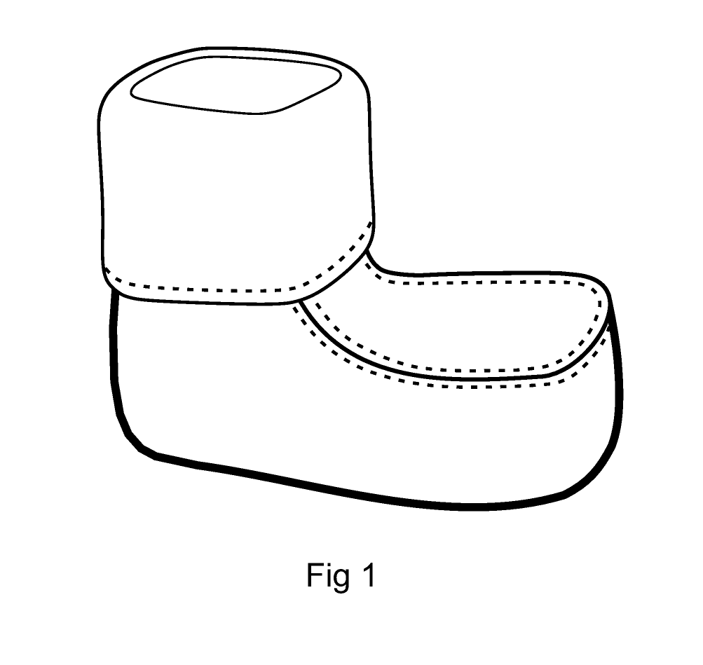

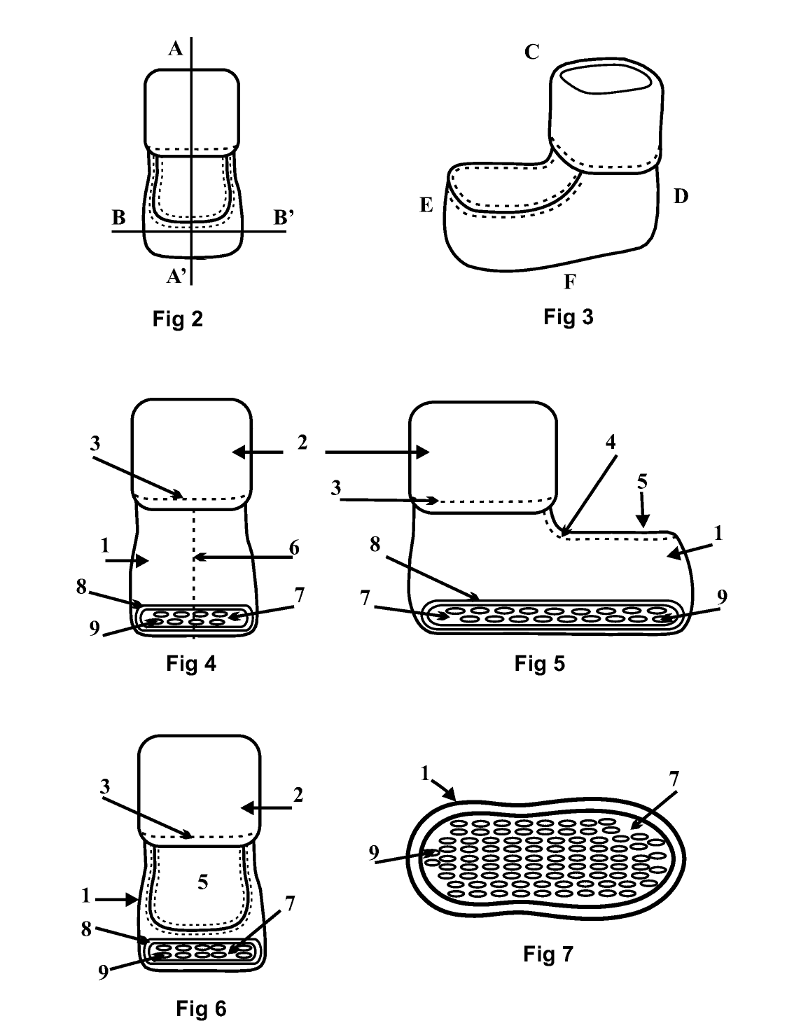

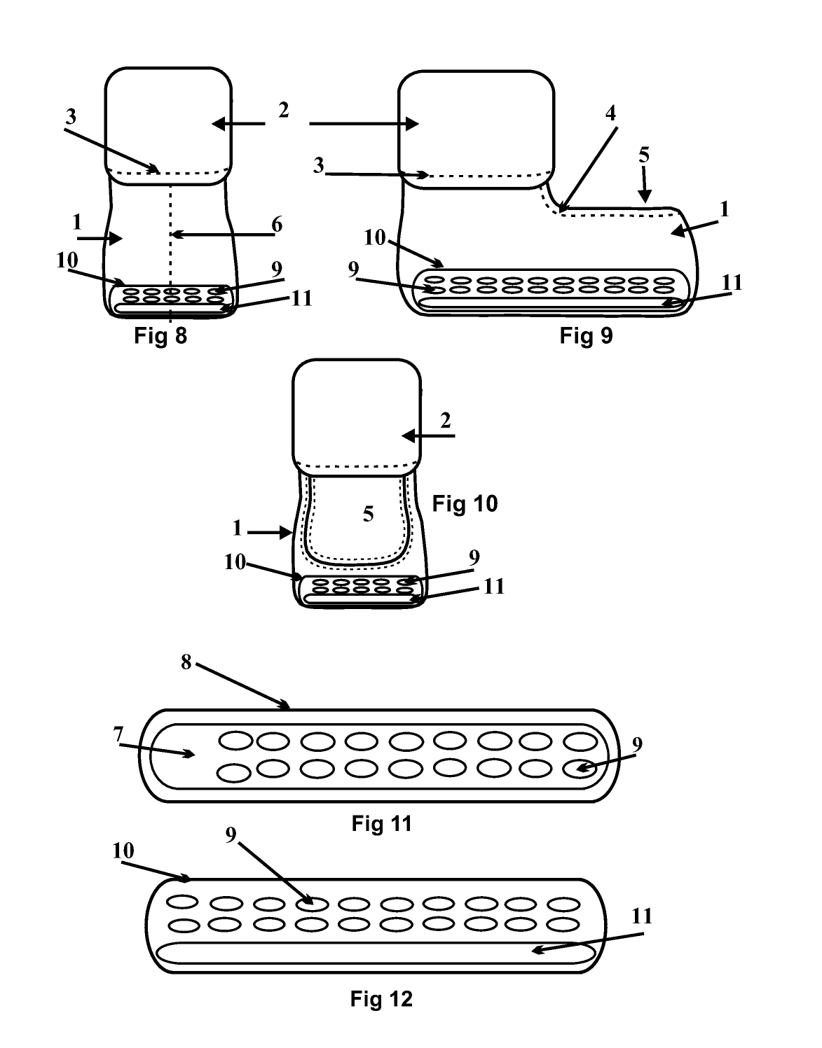

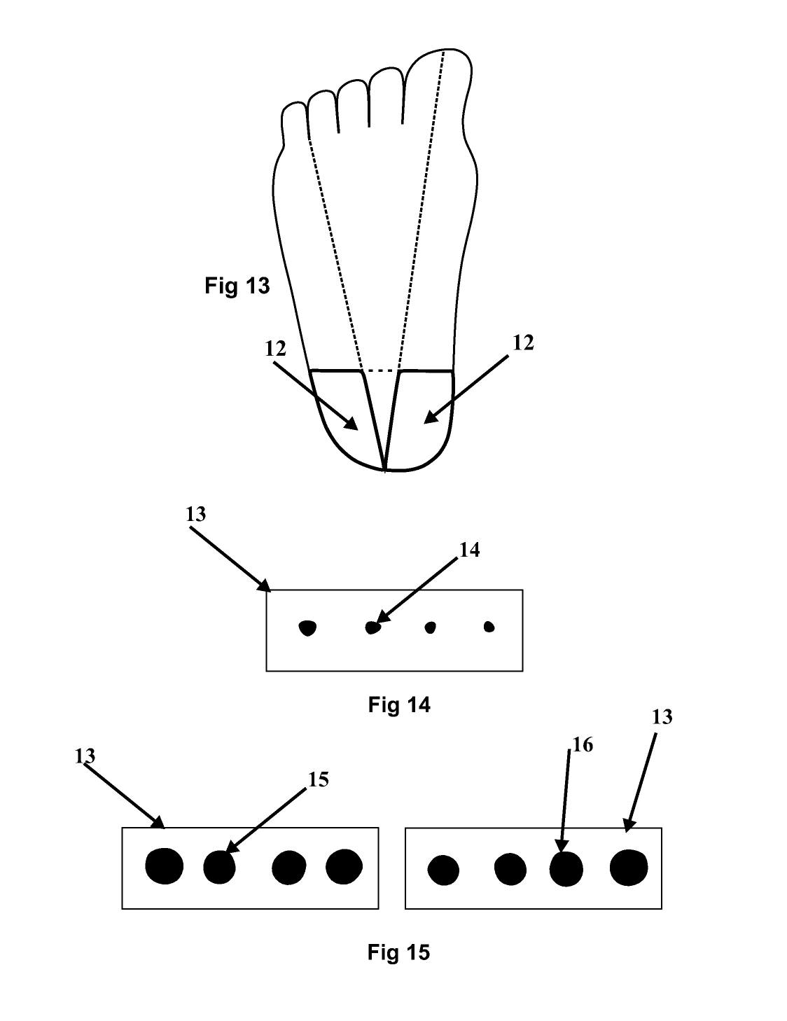

### USE OF THE BOOTY FOR BLOOD SAMPLING

In an example of use during the testing of the prototype, the booty is heated approximately 1 minute in the microwave device, the booty is placed on the foot of the newborn to be punctured and it is necessary to wait for between 10 and 20 minutes, while the mother nurses the baby if the extraction is destined to the neonatal research sampling, since in this case it is important that the baby has eaten food containing protein (breast milk, supplement with protein content).

The person taking the sample cleans his/her hands and makes sure to have the following materials available: alcohol, cotton, gloves, Lancet, leuco tape, support for drying of sample, and filter paper for deposit of the sample.

The person taking the sample ask the person who has the newborn in her arms to place the newborn in an upright position and remove the booty, the person taking the sample discards the booty into a container for disposable materials. The person taking the sample proceeds to perform the asepsis to all of the bottom part of the newborn's foot. The person taking the sample punctures the heel with a Lancet on the puncture area, cleans the heel to discard the interstitial fluid which arises immediately after the puncture, and leaves to form the blood drop completely. Takes the paper intended for the taking of the blood sample in such way that the end of the blood drop makes contact with it and allows to spread. The person taking the sample ensures that the drop went through the filter paper on both sides. The person taking the sample repeats the procedure to obtain at least three drops. The filter paper is allowed to dry between 4 and 6 hours.

If for any reason it is necessary to repeat the puncture. The person taking the sample uses another disposable heatable booty by repeating the procedure.

What is claimed is:

1. System to improve the quality of the sample of blood obtained from the foot of the infant and booty disposable Heatable characterized it because a booty sterile disposable Heatable manufactured in at least one type of fabric to cover the foot entirely and at least a fabric elastic for fit at the ankle, increases capillary action for glass expansion in the heel of the foot.

2. System to improve the quality of the sample of blood obtained from the foot of the infant and disposable spoils of the same according to the claim characterized 1 because no massage, rub or press the heel of the baby, transmitting heat without damaging nor burns on the delicate skin of the baby.

3. Booty disposable Heatable characterized by manufactured in at least one piece in fabric that can carry a seam or union to completely cover the foot, and a fist in fabric elastic that is sewn or attached to the workpiece mentioned above.

4. Booty disposable Heatable according to claim 3 which features at the bottom internal template of fabric wrapped in a soft fabric inside of which seeds are found on the fabric template.

5. Booty disposable Heatable according to claim 3 which features on the inside bottom wrapped in a soft fabric gel with seed capsule.
